SEQUENCE LISTING (17 sequences)

SEQ ID NO: 1 - protein, 13 aa, synthetic construct
AKFVAAWTLKAAA

SEQ ID NO: 2 - protein, 279 aa, synthetic construct
MFVFLVLLPLVSSVRFPNITNLCPFGEVFNATRFASVYAWNRKRISNCVADYSVLYNSASFSTFKCYGVSPTKLNDLCFTNVYADSFVIRGDEVRQIAPGQTGTIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPKKSTGVKLESMGIYQILAIYSTVASSLVLLVSLGAISFWMCSNGSLQCRICIAKFVAAWTLKAAA

SEQ ID NO: 3 - protein, 13 aa, Severe acute respiratory syndrome-related coronavirus 2
  SIGNAL 1..13
MFVFLVLLPLVSS

SEQ ID NO: 4 - protein, 205 aa, Severe acute respiratory syndrome-related coronavirus 2
VRFPNITNLCPFGEVFNATRFASVYAWNRKRISNCVADYSVLYNSASFSTFKCYGVSPTKLNDLCFTNVYADSFVIRGDEVRQIAPGQTGTIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPKKST

SEQ ID NO: 5 - protein, 10 aa, Alphainfluenzavirus influenzae
GVKLESMGIY

SEQ ID NO: 6 - protein, 38 aa, Alphainfluenzavirus influenzae
QILAIYSTVASSLVLLVSLGAISFWMCSNGSLQCRICI

SEQ ID NO: 7 - protein, 279 aa, synthetic construct
MFVFLVLLPLVSSVRFPNITNLCPFDEVFNATRFASVYAWNRKRISNCVADYSVLYNLAPFFTFKCYGVSPTKLNDLCFTNVYADSFVIRGDEVRQIAPGQTGNIADYNYKLPDDFTGCVIAWNSNKLDSKVSGNYNYLYRLFRKSNLKPFERDISTEIYQAGNKPCNGVAGFNCYFPLKSYSFRPTYGVGHQPYRVVVLSFELLHAPATVCGPKKSTGVKLESMGIYQILAIYSTVASSLVLLVSLGAISFWMCSNGSLQCRICIAKFVAAWTLKAAA

SEQ ID NO: 8 - protein, 205 aa, Severe acute respiratory syndrome-related coronavirus 2
VRFPNITNLCPFDEVFNATRFASVYAWNRKRISNCVADYSVLYNLAPFFTFKCYGVSPTKLNDLCFTNVYADSFVIRGDEVRQIAPGQTGNIADYNYKLPDDFTGCVIAWNSNKLDSKVSGNYNYLYRLFRKSNLKPFERDISTEIYQAGNKPCNGVAGFNCYFPLKSYSFRPTYGVGHQPYRVVVLSFELLHAPATVCGPKKST

SEQ ID NO: 9 - protein, 477 aa, synthetic construct
MFVFLVLLPLVSSVRFPNITNLCPFDEVFNATRFASVYAWNRKRISNCVADYSVLYNLAPFFTFKCYGVSPTKLNDLCFTNVYADSFVIRGDEVRQIAPGQTGNIADYNYKLPDDFTGCVIAWNSNKLDSKVSGNYNYLYRLFRKSNLKPFERDISTEIYQAGNKPCNGVAGFNCYFPLKSYSFRPTYGVGHQPYRVVVLSFELLHAPATVCGPKKSTGVKLESMGIYQILAIYSTVASSLVLLVSLGAISFWMCSNGSLQCRICIAKFVAAWTLKAAAGAAVYFLQSINFNAASPRWYFYYLNARLNEVAKNLKFIAGLIAIVNAAANFKDQVILLGAAAVLQSGFRKGAAAYYQLYSTQLNLITGRLQSLKLQLPQGTTLNAAKTFPPTEPKKAAGDAALALLLLNAAAMEVTPSGTWLGAGVAMPNLYKKAATLACFVLAAVNAAGLMWLSYFIGLWLLWPVTLNKQFDTYNLW

SEQ ID NO: 10 - protein, 195 aa, synthetic construct
VYFLQSINFNAASPRWYFYYLNARLNEVAKNLKFIAGLIAIVNAAANFKDQVILLGAAAVLQSGFRKGAAAYYQLYSTQLNLITGRLQSLKLQLPQGTTLNAAKTFPPTEPKKAAGDAALALLLLNAAAMEVTPSGTWLGAGVAMPNLYKKAATLACFVLAAVNAAGLMWLSYFIGLWLLWPVTLNKQFDTYNLW

SEQ ID NO: 11 - RNA, 35 nt, synthetic construct
  source 1..35: mol_type = other RNA
cctgaatggactacgacatagtctagtccgccaag

SEQ ID NO: 12 - RNA, 44 nt, synthetic construct
  source 1..44: mol_type = other RNA
ataggcggcgcatgagagaagcccagaccaattacctacccaaa

SEQ ID NO: 13 - RNA, 125 nt, synthetic construct
  source 1..125: mol_type = other RNA
gcgatcgcatacagcagcaattggcaagctgcttacatagaactcgcggcgattggcatgccgccttaaaatttttattttatttttcttttcttttccgaatcggattttgtttttaatatttc

SEQ ID NO: 14 - RNA, 55 nt, synthetic construct
  source 1..55: mol_type = other RNA
aaaaaaaaaaaaaaaaaaaaaaaaaaaaaaaaaaaaaaaaaaaaaaaaaaaaaaa

SEQ ID NO: 15 - protein, 2493 aa, Venezuelan equine encephalitis virus
MEKVHVDIEEDSPFLRALQRSFPQFEVEAKQVTDNDHANARAFSHLASKLIETEVDPSDTILDIGSAPARRMYSKHKYHCICPMRCAEDPDRLYKYATKLKKNCKEITDKELDKKMKELAAVMSDPDLETETMCLHDDESCRYEGQVAVYQDVYAVDGPTSLYHQANKGVRVAYWIGFDTTPFMFKNLAGAYPSYSTNWADETVLTARNIGLCSSDVMERSRRGMSILRKKYLKPSNNVLFSVGSTIYHEKRDLLRSWHLPSVFHLRGKQNYTCRCETIVSCDGYVVKRIAISPGLYGKPSGYAATMHREGFLCCKVTDTLNGERVSFPVCTYVPATLCDQMTGILATDVSADDAQKLLVGLNQRIVVNGRTQRNTNTMKNYLLPVVAQAFARWAKEYKEDQEDERPLGLRDRQLVMGCCWAFRRHKITSIYKRPDTQTIIKVNSDFHSFVLPRIGSNTLEIGLRTRIRKMLEEHKEPSPLITAEDIQEAKCAADEAKEVREAEELRAALPPLAADFEEPTLEADVDLMLQEAGAGSVETPRGLIKVTSYAGEDKIGSYAVLSPQAVLKSEKLSCIHPLAEQVIVITHSGRKGRYAVEPYHGKVVVPEGHAIPVQDFQALSESATIVYNEREFVNRYLHHIATHGGALNTDEEYYKTVKPSEHDGEYLYDIDRKQCVKKELVTGLGLTGELVDPPFHEFAYESLRTRPAAPYQVPTIGVYGVPGSGKSGIIKSAVTKKDLVVSAKKENCAEIIRDVKKMKGLDVNARTVDSVLLNGCKHPVETLYIDEAFACHAGTLRALIAIIRPKKAVLCGDPKQCGFFNMMCLKVHFNHEICTQVFHKSISRRCTKSVTSVVSTLFYDKRMRTTNPKETKIVIDTTGSTKPKQDDLILTCFRGWVKQLQIDYKGNEIMTAAASQGLTRKGVYAVRYKVNENPLYAPTSEHVNVLLTRTEDRIVWKTLAGDPWIKILTAKYPGNFTATIEEWQAEHDAIMRHILERPDPTDVFQNKANVCWAKALVPVLKTAGIDMTTEQWNTVDYFETDKAHSAEIVLNQLCVRFFGLDLDSGLFSAPTVPLSIRNNHWDNSPSPNMYGLNKEVVRQLSRRYPQLPRAVATGRVYDMNTGTLRNYDPRINLVPVNRRLPHALVLHHNEHPQSDFSSFVSKLKGRTVLVVGEKLSVPGKKVDWLSDQPEATFRARLDLGIPGDVPKYDIVFINVRTPYKYHHYQQCEDHAIKLSMLTKKACLHLNPGGTCVSIGYGYADRASESIIGAIARQFKFSRVCKPKSSHEETEVLFVFIGYDRKARTHNPYKLSSTLTNIYTGSRLHEAGCAPSYHVVRGDIATATEGVIINAANSKGQPGGGVCGALYKKFPESFDLQPIEVGKARLVKGAAKHIIHAVGPNFNKVSEVEGDKQLAEAYESIAKIVNDNNYKSVAIPLLSTGIFSGNKDRLTQSLNHLLTALDTTDADVAIYCRDKKWEMTLKEAVARREAVEEICISDDSSVTEPDAELVRVHPKSSLAGRKGYSTSDGKTFSYLEGTKFHQAAKDIAEINAMWPVATEANEQVCMYILGESMSSIRSKCPVEESEASTPPSTLPCLCIHAMTPERVQRLKASRPEQITVCSSFPLPKYRITGVQKIQCSQPILFSPKVPAYIHPRKYLVETPPVEETPESPAENQSTEGTPEQPALVNVDATRTRMPEPIIIEEEEEDSISLLSDGPTHQVLQVEADIHGSPSVSSSSWSIPHASDFDVDSLSILDTLDGASVTSGAVSAETNSYFARSMEFRARPVPAPRTVFRNPPHPAPRTRTPPLAHSRASSRTSLVSTPPGVNRVITREELEALTPSRAPSRSASRTSLVSNPPGVNRVITREEFEAFVAQQQXRFDAGAYIFSSDTGQGHLQQKSVRQTVLSEVVLERTELEISYAPRLDQEKEELLRKKLQLNPTPANRSRYQSRRVENMKAITARRILQGLGHYLKAEGKVECYRTLHPVPLYSSSVNRAFSSPKVAVEACNAMLKENFPTVASYCIIPEYDAYLDMVDGASCCLDTASFCPAKLRSFPKKHSYLEPTIRSAVPSAIQNTLQNVLAAATKRNCNVTQMRELPVLDSAAFNVECFKKYACNNEYWETFKENPIRLTEENVVNYITKLKGPKAAALFAKTHNLNMLQDIPMDRFVMDLKRDVKVTPGTKHTEERPKVQVIQAADPLATADLCGIHRELVRRLNAVLLPNIHTLFDMSAEDFDAIIAEHFQPGDCVLETDIASFDKSEDDAMALTALMILEDLGVDAELLTLIEAAFGEISSIHLPTKTKFKFGAMMKSGMFLTLFVNTVINIVIASRVLRERLTGSPCAAFIGDDNIVKGVKSDKLMADRCATWLNMEVKIIDAVVGEKAPYFCGGFILCDSVTGTACRVADPLKRLFKLGKPLAVDDEHDDDRRRALHEESTRWNRVGILPELCKAVESRYETVGTSIIVMAMTTLASSVKSFSYLRGAPITLYG

SEQ ID NO: 16 - protein, 266 aa, synthetic construct
MFVFLVLLPLVSSVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTGVKLESMGIYQILAIYSTVASSLVLLVSLGAISFWMCSNGSLQCRICI

SEQ ID NO: 17 - protein, 205 aa, Severe acute respiratory syndrome-related coronavirus 2
VRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKST

(19) United States
(12) Patent Application Publication
(10) Pub. No.: US 2023/0201333 A1
(43) Pub. Date: Jun. 29, 2023

(54) EFFICIENT VACCINE

(71) Applicant: VLP Therapeutics Japan, LLC, Tokyo (JP)

(72) Inventors: Wataru AKAHATA, Kensington, MD (US); Jonathan F. SMITH, Redwood City, CA (US); Jeffery Leo ALEXANDER, San Diego, CA (US); Takashi SEKIDA, Nishinomiya-shi (JP)

(73) Assignee: VLP Therapeutics Japan, LLC, Tokyo (JP)

(21) Appl. No.: 18/067,358

(22) Filed: Dec. 16, 2022

Related U.S. Application Data
(60) Provisional application No. 63/265,634, filed on Dec. 17, 2021.
(60) Provisional application No. 63/393,400, filed on Jul. 29, 2022.

(51) Int. Cl. A61K 39/215; A61P 31/14

(52) U.S. Cl. CPC A61K 39/215; A61P 31/14; A61K 2039/53

(57) ABSTRACT

Provided herein is an isolated polynucleotide, which encodes structural proteins nsp1, nsp2, nsp3 and nsp4 and a polypeptide comprising an antigenic protein fused to a signal sequence, a transmembrane domain and at least one peptide selected from CD4+ T cell epitopes and CD8+ T cell epitopes. The polynucleotide is useful for manufacturing a vaccine against virus infection, especially, COVID-19 infection, the treatment of a cancer and/or an inflammatory disease.

### CROSS REFERENCE TO RELATED APPLICATIONS

This application claims the benefit of U.S. Provisional Patent Application No. 63/265,634 filed on Dec. 17, 2021 and U.S. Provisional Patent Application No. 63/393,400 filed on Jul. 29, 2021. The entire disclosures of those prior applications are hereby incorporated by reference.

### INCORPORATION BY REFERENCE OF SEQUENCE LISTING

The content of the electronically submitted sequence listing, file name: Q282254_Sequence_Listing_as_Filed.xml; size: 20,919 bytes; and date of creation: Dec. 16, 2022, filed herewith, is incorporated herein by reference in its entirety.

### TECHNICAL FIELD

The present disclosure relates generally to the field of an efficient vaccine comprising an alpha virus replicon and antigen and to a method and a composition for treating and/or immunizing against antigens. In particular, the present disclosure relates to a vaccine for coronavirus such as SARS-CoV-2 (COVID-19).

### BACKGROUND ART

Coronaviruses are a large family of viruses that usually cause mild to moderate upper-respiratory tract illnesses, like the common cold. However, three new coronaviruses have emerged from animal reservoirs over the past two decades to cause serious and widespread illness and death.

There are hundreds of coronaviruses, most of which circulate among such animals as pigs, camels, bats and cats. Sometimes those viruses jump to humans-called a spillover event- and can cause disease. Four of the seven known coronaviruses that sicken people cause only mild to moderate disease. Three can cause more serious, even fatal, disease. SARS coronavirus (SARS-CoV) emerged in November 2002 and caused severe acute respiratory syndrome (SARS). That virus disappeared by 2004. Middle East respiratory syndrome (MERS) is caused by the MERS coronavirus (MERS-CoV). Transmitted from an animal reservoir in camels, MERS was identified in September 2012 and continues to cause sporadic and localized outbreaks. The third novel coronavirus to emerge in this century is called SARS-CoV-2. It causes coronavirus disease 2019 (COVID-19), which emerged from China in December 2019 and was declared a global pandemic by the World Health Organization on Mar. 11, 2020. (Coronaviruses: https://www.niaid.nih.gov/diseases-conditions/coronaviruses)

Most people infected with the SARS-CoV-2 virus will experience mild to moderate respiratory illness and recover without requiring special treatment. Older people, and those with underlying medical problems like cardiovascular disease, diabetes, chronic respiratory disease, and cancer are more likely to develop serious illness. Anyone can get sick with COVID-19 and become seriously ill or die at any age. (Coronavirus disease (COVID-19): https://www.who.int/health-topics/coronavirus #tab=tab_1)

Omicron (B.1.1.529 variant) was first reported to WHO from South Africa as a new SARS-CoV-2 variant on 24 Nov. 2021.

Preliminary evidence suggests there may be an increased risk of reinfection with Omicron (ie, people who have previously had COVID-19 could become reinfected more easily with Omicron), as compared to other variants of concern. (Update on Omicron: https://www.who.int/news/item/28-11-2021-update-on-omicron)

There are currently several approved vaccines against COVID-19. In particular, mRNA vaccine shows over 95% efficacy. However, it is needed to provide more vaccines to worldwide to prevent this pandemic. To provide the vaccine efficient even for any variant to worldwide, more vaccine development is needed, in particular the vaccine based on the technologies which enable to produce vaccine efficient for any variant in short time.

### PATENT LITERATURE

### [PTL 1]

WO2021/21068

### SUMMARY OF INVENTION

An object of the present disclosure is to provide a new design concept for developing a vaccine against an antigen such as a viral antigen, cancer antigen and others which is effective for preventing or treating an infectious disease or a cancer.

The present disclosure relates to novel antigenically-active proteins/polypeptides capable of inducing immune responses against antigens. The protein/polypeptide disclosed herein include an antigenic protein fused to a signal sequence, transmembrane domain and at least one peptide selected from CD4+ T cell epitopes and CD8+ T-cell epitopes. The antigenic protein may be a protein derived from a coronavirus structural protein or a fragment thereof.

The coronavirus structural proteins may comprise spike(S) protein, nucleocapsid (N) protein, membrane (M) protein and a small envelope protein (E). The antigenic protein may be derived from any of the structural proteins or a combination thereof. Specific examples of the antigenic proteins may include S1 and/or S2 subunit of the spike protein and especially, receptor binding domain (RBD) of the S1 subunit.

In another aspect, the present disclosure relates to a novel polynucleotide encoding the above discussed novel antigenically-active proteins/polypeptides which is capable of inducing protection against the antigenic protein.

In another aspect, the present disclosure relates to a novel alphavirus replicon that can express the above discussed antigenically-active protein/polypeptide. The alphavirus replicon includes polynucleotide such as RNA encoding alphavirus non-structural proteins nsp1, nsp2, nsp3 and nsp4 and a polynucleotide encoding the above-discussed antigenically active protein/polypeptide as a gene of interest.

In yet another aspect, the present disclosure relates to a vaccine comprising the above discussed polypeptide or polynucleotide. Especially, the present disclosure provides a vaccine comprising a polynucleotide encoding alphavirus non-structural proteins nsp1, nsp2, nsp3 and nsp4, and a polypeptide comprising an antigenic protein fused to a signal sequence, transmembrane domain and at least one peptide selected from CD4+ T cell epitopes and CD8+ T cell epitopes. The CD4+ T cell epitope may be a universal epitope. In the case of the antigenic protein is a protein derived from a viral structural protein, the vaccine can be used for preventing and/or treating a subject from virus infection.

In yet another aspect, the present disclosure relates to a method for immunizing, preventing or treating a subject from virus infection comprising administering an effective amount of the above-discussed polypeptide or polynucleotide to the subject in need thereof.

In still another aspect, the present disclosure relates to use of the above-discussed polypeptide or polynucleotide for the manufacture of a medicament.

In further aspect, the present disclosure relates to a novel polynucleotide, which encodes alphavirus non-structural proteins nsp1, nsp2, nsp3 and nsp4 and a polypeptide comprising an antigenic peptide wherein the polynucleotide comprises a modified nucleoside (e.g. N1-methyl-pseudouridine or 5-methyl-cytidine or both).

This efficient vaccine design is also applied for developing vaccines for the treatment of a cancer or an inflammatory disease.

### BRIEF DESCRIPTION OF DRAWINGS

FIG. 1

A construct of an alphavirus replicon.

FIG. 2

ELISA against SARS-CoV-2 variant's RBD proteins.

FIG. 3

ACE Inhibition Assay

FIG. 4

Effect of the polynucleotide with a modified nucleoside in innate immunity.

FIG. 5

Effect of the vaccine compositions on the body weights of hamsters challenged with COVID-19 WT strain or gamma variant.

FIG. 6

Effect of the vaccine compositions on the Quantitative RT-PCR Assay for SARS-CoV-2 in hamsters challenged with SARS-COV-2 WT strain or Gamma variant

FIG. 7

Effect of the vaccine composition on histopathologic analysis of the lung of the hamsters challenged with SARS-COV-2 WT strain or Gamma variant.

### DESCRIPTION OF EMBODIMENTS

As used herein, “antigen” refers to a molecule capable of being bound by an antibody or a T cell receptor. An antigen is additionally capable of being recognized by the immune system and/or being capable of inducing a humoral immune response and/or cellular immune response leading to the activation of B- and/or T-lymphocytes. Antigens, as used herein, include but are not limited to virus, allergens, self-antigens, haptens, cancer antigens (i.e. tumor antigens) and infectious disease antigens such as Mycobacterium tuberculosis as well as small organic molecules such as drugs of abuse (like nicotine) and fragments and derivatives thereof. Furthermore, antigens used for the present disclosure can be peptides, proteins, domains, carbohydrates, alkaloids, lipids or small molecules such as, for example, steroid hormones and fragments and derivatives thereof, autoantibody and cytokine itself. As used herein, “antigenic peptide” refers to a protein or peptide which can act as an antigen.

As used herein, “virus” may be Severe acute respiratory syndrome-related coronavirus (SARS), severe acute respiratory syndrome coronavirus 2 (SARS-CoV-2), Ebola, HIV, Hepatitis B virus (HBV), influenza, Hepatitis C virus (HCV), Human papillomavirus (HPV), Cytomegalovirus (CMV), Chikungunya virus, Respiratory syncytial virus (RSV), Dengue virus, or a orthymyxoviridae family virus, but not limited thereto.

As used herein, “coronavirus” is meant to refer to single-stranded, positive-sense RNA viruses that belong to the family, Coronaviridae. Exemplary Coronaviridae viruses include but are not limited to SARS-Cov, MERS-Cov and SARS-CoV-2 (COVID-19). SARS-CoV-2 (COVID-19) may include known and unknown mutants. Known mutants may include SARS-CoV-2 E484K_N501Y_K417T mutant (Gamma, Brazilian valiant), T478K_L452R (Delta, Indian valiant), E484K_N501Y_K417N mutant (Beta, South African variant), E484K mutant and K417N_S477N_T478K_E484A_Q493K_G446S_N501Y_Y505H_G496S_Q498R_N440K_G339D_S375F_S373P_S371L mutant (Omicron). The coronavirus genome encodes numerous non-structural proteins and four major structural proteins including the spike (S), nucleocapsid (N), membrane (M) and small envelope (E) proteins. Spike (S) protein, a large envelope glycoprotein, is composed of S1 and S2 subunits. The “Receptor-binding domain (RBD)” is located in the S1 subunit. Preferable example of the antigenic protein used herein is coronavirus RBD, a fragment thereof or a mutant thereof. Spike protein, S1 and S2 subunits of the spike protein and RBD of COVID-19 and its mutants have been identified and published (Wrapp et al., Science Vol. 367, issue 6483(2020) pp. 1260-1263 10.1126/science.abb2507, Z. Wang et al., Nature Vol 592(2021) pp 616-622 the contents of the cited documents are incorporated herein by reference).

“Coronavirus structural protein” used herein may be a naturally occurring virus structural protein or a modified protein thereof. The modified protein may be a fragment of the naturally occurring virus structural protein or its mutant. In one embodiment, the modified protein has at least 70%, 75%, 80%, 85%, 90%, 95% or 98% amino acid sequence identity to a naturally occurring viral structural protein or its fragment. In one embodiment, the modified protein is a mutant where at most 10% of the amino acids are deleted, substituted, and/or added based on a naturally occurring viral envelope protein or its fragment.

As used herein, “transmembrane domain (TM)” is a protein derived either from a natural or from a synthetic source. Where the source is natural, the domain in some aspects is derived from any membrane-bound or transmembrane protein. In one aspect, the membrane-bound or transmembrane protein is a protein heterologous to the origin of the antigenic peptide. Examples of the membrane-bound or transmembrane proteins may include the alpha, beta or zeta chain of a T-cell receptor, CD28, CD3 epsilon, CD45, CD4, CD5, CDS, CD9, CD16, CD22, CD33, CD37, CD64, CD80, CD86, CD134, CD137, CD154; toll-like receptors (TLR) such as TLR1-TLR10 in human and TLR1-TLR9, TLR11-TLR13 in mouse; interleukin (IL) receptors such as IL-1-28 receptor, RANTES receptors (CCR1, CCR3, CCR5), MIP-1 receptor, PF4 receptor, M-CSF receptor and NAP-2 receptor belonging to GPCR chemokine receptor; hemagglutinin (HA). In another aspect, the membrane-bound or transmembrane protein is a protein derived from COVID-19, such as COVID-19 spike protein.

Examples of transmembrane proteins may also include the followings:
5-Lipoxygenase-Activating Protein, ABC Transporters, ACBP, Amyloid beta (A4), Bcl-2 Inhibitors, BNIPs, CAAX protease, Cytochromes P450, E-NPPs, EPHA1, EPHA2, EPHA3, EPHA4, Fatty Acid Desaturases, Gamma secretase, Glucose transporter, Glycophorins, GPCR, HER2/ErbB2, HER3/ErbB3, HER4/ErbB4, HSD-11p, Hypoxia-induced Proteins, Immunoglobulins, Insulin receptor, Integrins, Ion channel, MAPEG, MFS, MinK Family, MPPs, Peptidase AD, Peptidase Family M48, Peptidase MA, Protein Jagged, Receptor-type Kinases, SNARE Complex, Sulfatases, TNF receptor, Transmembrane Proteins 14, Transporter, TROBP, VEGF receptors, Aldehyde Dehydrogenases, Ammonia and Urea transporters, FMN-linked Oxidoreductases, Leucine Rich Repeat (LRR)-Containing Transmembrane Proteins, Leukotriene C4 synthase, Lysosome-associated membrane glycoprotein, Major Intrinsic Protein (MIP)/FNT superfamily, Microsomal prostaglandin E synthase, N-(deoxy)ribosyltransferase-like Membrane Proteins, Neutral/alkaline Ceramidases, Oligosaccharyl Transferase, Pentameric Ligand-gated Ion Channels, Rhodopsin-like receptors and pumps, Single-helix ATPase Regulators, Squalene/phytoene Synthase, Stearoyl-CoA desaturase 1, Stannin (SNN) Membrane Proteins, T-cell Surface Glycoprotein CD3 Zeta Chain, Tetratricopeptide repeat (TPR) Alpha-Helical Repeat Proteins, Transmembrane Proteins with NAD(P)-binding Rossmann-fold Domains.

In addition, monotypic/peripheral proteins that are attached to the lipid bilayer or other integral proteins and peptide may also be used as transmembrane proteins. Examples may include Alpha/Beta-Hydrolase, Annexins, Bet V1-Like Protein, C1 Domain-Containing Protein, C2 Domain-containing Protein, CoA-Dependent Acyltransferases, CRAL-TRIO Domain-Containing Protein, DNase I-like protein, Fibrinogen, FYVE/PHD Zinc Finger Protein, Galactose-Binding Domain-Like Protein, Glycolipid Transfer Protein, Immunoglobulin-Like Superfamily (E Set) Protein, Lipocalin, Lipoxygenase, PGBD superfamily, PH Domain-Like Protein, Phosphatidylinositol 3-/4-Kinase, PLC-like Phosphodiesterase, Phosphotyrosine Protein Phosphatases II, P-Loop Containing Nucleoside Triphosphate Hydrolase, Protein kinase superfamily, PX Domain-Containing Protein, Saposin, Synuclein and Transcriptional factor tubby.

In one aspect of the present disclosure, the transmembrane domain includes at least transmembrane region(s) of the membrane-bound or transmembrane protein. In addition, the transmembrane domain may also include juxtamembrane domain (JMD) and/or cytoplasmic tail of the membrane-bound or transmembrane protein.

Alternatively, the transmembrane domain in some embodiments is synthetic. In some aspects, the synthetic transmembrane domain comprises predominantly hydrophobic residues such as leucine and valine. In some aspects, a triplet of phenylalanine, tryptophan and valine will be found at each end of a synthetic transmembrane domain.

Preferable transmembrane domain may be those derived from influenza virus hemagglutinin (HA), CD80, Toll-like receptor 4(TLR4) or COVID-19 spike protein. Specific examples may include a protein comprising the flexible juxtamembrane region or flexible linker, the transmembrane domain and the cytoplasmic tail of Influenza virus hemagglutinin “HA (flexible-TM-Cyt)”; a protein consisting of transmembrane domain and cytoplasmic tail of human CD80; a protein consisting of transmembrane domain (TM) and Toll/interleukin-1 receptor domain (TIR), and a protein consisting of the juxtamembrane domain (JMD) and transmembrane domain of COVID-19 Spike (S) protein.

As used herein, “nucleoside” refers to a molecule consisting of a guanine (G), adenine (A), thymine (T), uridine (U), cytidine (C) or a modified nucleoside thereof.

A modified nucleoside includes, but not limited to, pseudouridine, N1-methyl-pseudouridine, 5-methyl-uridine, pseudoucytidine, N1-methyl-pseudocytidine and 5-methyl-cytidine.

Pseudouridine or pseudocytidine is an isomer of the uridine or cytidine in which the uracil or cytosine is attached via a carbon-carbon instead of a nitrogen-carbon glycosidic bond.

In one embodiment, the modified nucleoside is independently selected from N1-methyl-pseudouridine or 5-methyl-cytidine.

In one embodiment, substantially 100% of cytidine and uridine included in mRNA or saRNA are modified cytidine (e.g. 5-methyl-cytidine) and modified uridine (e.g. N1-methyl-pseudouridine) respectively. In one embodiment, 100% of cytidine is modified cytidine and 80% uridine is modified uridine, and in another embodiment, 50% of cytidine is modified cytidine and 50% of uridine is modified uridine. In yet another embodiment, less than 100% uridine in a saRNA is modified uridine.

As used herein, “signal sequence” (sometimes referred to as signal peptide, targeting signal, localization signal, localization sequence, transit peptide, leader sequence or leader peptide) is a polynucleotide or polypeptide, depending on the context. Signal sequence: is from about 9 to 200 nucleotides or 3-70 amino acids in length that, optionally, is incorporated at the 5′ or N-terminus of the coding region or a protein. Some signal sequences are cleaved from the protein, for example by a signal peptidase after the proteins are transported to the desired site.

In some embodiments, the si al sequence of IL-2, especially human IL-2 may be employed. In another embodiments, the signal sequence of the COVID-19 spike protein may be employed.

As used herein, CD4+ T-cell epitope is a peptide that binds to a major histocompatibility complex (MHC) class II molecule (MHC-II) and triggers a CD4+ T cell immune response. The CD4+ T cell epitope may be a peptide derived from the virus from which the antigenic protein is derived. CD4+ T cell epitopes derived from the viral proteins can be identified by a known epitope-mapping procedure. In the present disclosure, a peptide consisting of multiple CD4+ T-cell epitopes connected by a linker such as “AA” may be used as “CD4+ T cell epitope”. Alternatively, CD4+ T cell epitope may be a Pan-DR epitope (PADRE).

The expression “PADRE” used in the present disclosure means Pan HLA DR-binding epitope or a universal epitope, a peptide that activates almost all CD4+ T cells irrespective of the HLA haplotypes. Various peptides have been identified as PADRE and any of the known PADRE may be used in this disclosure. One example of PADRE is AKFVAAWTLKAAA (SEQ ID NO: 1).

As used herein, CD8+ T cell epitope is a peptide that binds to major histocompatibility complex (MHC) class I molecule (MHC-I) and triggers a CD8+ T cell immune response. The CD8 T cell epitope may be a peptide derived from the virus from which the antigenic protein is derived. CD8+ T cell epitopes derived from the viral proteins can be identified by a known epitope-mapping procedure. In the present disclosure, a peptide consisting of multiple CD8+ T-cell epitopes connected by a linker such as AA may be used as “CD8+ T cell epitope”.

The antigenic protein, a signal sequence, a transmembrane domain and at least one peptide selected from CD4+ T cell epitopes and CD8+ T cell epitopes may be directly or indirectly fused. In one embodiment, one or two linkers may intervene between them.

Also, the coronavirus structural protein, a signal sequence, a transmembrane domain and at least one peptide selected from CD4+ T cell epitopes and CD8+ T cell epitopes can be truncated and replaced by short linkers. In some embodiments, the coronavirus structural protein, the transmembrane domain and/or the signal sequence include one or more peptide linkers.

An example of a short linker consists of from 2 to 25 amino acids (e.g. 2, 3, 4, 5 or 6 amino acids). Usually, it is from 2 to 15 amino acids in length, such as SG, GS, SGG, GGS SGSG and TRGGS. In certain circumstances, the linker can consist of only one amino acid, such as glycine (G), serine (S) and cysteine (C).

When the coronavirus structural protein is chemically conjugated to a signal sequence, a transmembrane domain and at least one peptide selected from CD4+ T cell epitopes and CD8+ T cell epitopes through a chemical cross-linker, examples of the cross-linkers include, but are not limited to, SMPH, Sulo-MBS, Sulfo-EMCS, Sulfo-GMBS, Sulfo-SIAB, Sulfo-SMPB, Sulfo-SMCC, SVSB, and STA. Chemical cross-linkers available on the market may also be employed.

IgG-derived substances can also be used as a linker. Examples of IgG-derived substances include IgG1 to IgG4 comprising (i) full (hinge-CH₂CH₃) (ii) half (hinge-CH₃) and (iii) short (12aa hinge only). Preferable example is IgG4-CH₃.

As used herein “alphavirus” is meant to refer to RNA-containing viruses that belong to the Togaviridae family of viruses. Exemplary Togaviridae viruses include but are not limited to Eastern Equine Encephalitis Virus (EEEV), Venezuelan. Equine Encephalitis Virus (VEEV), Everglades Virus, Mucambo Virus, Pixuna Virus, Western Equine Encephalitis Virus (WEEV), Sindbis Virus, Semliki Forest Virus, Middleburg Virus, Chikungunya Virus (CHIKV), O'nyong-nyong Virus, Ross River Virus, Barmah Forest Virus, Getah Virus, Sagiyama Virus, Bebaru Virus, Mavaro Virus, Una Virus, Aura Virus, Whataroa Virus, Babanki Virus, Kyzylagach Virus, Highlands J virus, Fort Morgan Virus, Ndumu Virus, Buggy Creek Virus and Ockelbo virus.

By “alphavirus structural protein” is meant a polypeptide or fragment thereof having at least about 80% amino acid sequence identity to a naturally occurring viral capsid or envelope protein. In one embodiment, the alphavirus structural protein has at least about 85%, 90%, 95% or greater amino acid sequence identity with Eastern Equine Encephalitis Virus (EEEV), Venezuelan Equine Encephalitis Virus (VEEV), Everglades Virus, Mucambo Virus, Pixuna Virus, Western Equine Encephalitis Virus (WEEV), Sindbis Virus, Semliki Forest Virus, Middleburg Virus, Chikungunya Virus (CHIKV), O'nyong-nyong Virus, Ross River Virus, Barmah Forest Virus, Getah Virus, Sagiyama Virus, Bebaru Virus, Mayaro Virus, Una Virus, Aura Virus, Whataroa Virus, Babanki Virus, Kyzylagach Virus, Highlands J virus, Fort Morgan Virus, Ndumu Virus, or Buggy Creek Virus. Wild type amino acid sequences of alphavirus structural proteins can be obtained from GenBank.

In specific embodiments, the alphavirus is a CHIKV, for example CHIKV strain 37997 or LR2006 OPY-1. In other embodiments, the alphavirus is a VEEV for example VEEV strain TC-83.

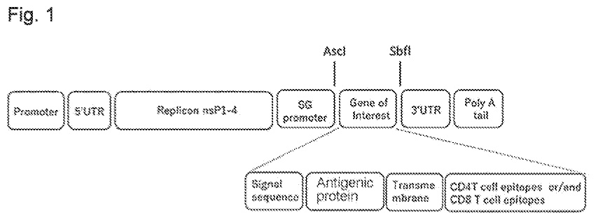

By “an alphavirus replicon” is meant an RNA molecule which can direct its own amplification in vivo in a target cell. The replicon encodes the polymerase (s) which catalyze RNA amplification (nsp1, nsp2, nsp3, nsp4) and contains cis RNA sequences required for replication which are recognized and utilized by the encoded polymerase(s). An alphavirus replicon typically contains the following ordered elements: 5′ UTR, sequences which encode alphavirus nonstructural proteins (nsp1, nsp2, nsp3, nsp4), 3′ UTR, and a poly A tail. An alphavirus replicon also contains one Cr more viral sub-genomic (SG) promoters directing the expression of the gene of interest. Those sequences may have one or more mutations taught in a Prior art. The alphavirus replicon provided by the present disclosure may have the construct shown in FIG. 1.

In this disclosure, “comprises,” “comprising,” “containing” and “having” and the like can have the meaning ascribed to them in U.S. Patent law and can mean “includes,” “including,” and the like; “consisting essentially of or “consists essentially” likewise has the meaning ascribed in U.S. Patent law and the term is open-ended, allowing for the presence of more than that which is recited so long as basic or novel characteristics of that which is recited is not changed by the presence of more than that which is recited, but excludes prior art embodiments.

By “fragment” is meant a portion of a protein, polypeptide or polynucleotide. This portion contains, preferably, at least 10%, 20%, 30%, 40%, 50%, 60%, 70%, 80%, or 90% of the entire length of the reference nucleic acid molecule or polypeptide. A fragment may contain 10, 20, 30, 40, 50, 60, 70, 80, 90, or 100, 200, 300, 400, 500, 600, 700, 800, 900, or 1000 nucleotides or amino acids.

By “reference” is meant a standard or control condition.

A “reference sequence” is a defined sequence used as a basis for sequence comparison. A reference sequence may be a subset of or the entirety of a specified sequence; for example, a segment of a full-length cDNA or gene sequence, or the complete cDNA or gene sequence. For polypeptides, the length of the reference polypeptide sequence will generally be at least about 16 amino acids, preferably at least about 20 amino acids, more preferably at least about 25 amino acids, and even more preferably about 35 amino acids, about 50 amino acids, or about 100 amino acids. For nucleic acids, the length of the reference nucleic acid sequence will generally be at least about 50 nucleotides, preferably at least about 60 nucleotides, more preferably at least about 75 nucleotides, and even more preferably about 100 nucleotides or about 300 nucleotides or any integer thereabout or there between.

Sequence identity is typically measured using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions, deletions, and/or other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine. In an exemplary approach to determining the degree of identity, a BLAST program may be used, with a probability score between e<″3> and e<″100> indicating a closely related sequence.

By “effective amount” is meant the amount of an agent required to ameliorate the symptoms of a disease relative to an untreated patient. The effective amount of active compound(s) used to practice the present disclosure for prevention or treatment of a disease varies depending upon the manner of administration, the age, body weight, and general health of the subject. Ultimately, the attending physician or veterinarian will decide the appropriate amount and dosage regimen. Such amount is referred to as an “effective” amount.

A satisfactory effect can be obtained by systemic administration, e.g. intramuscular administration, subcutaneous administration or intravenous administration 1-4 times at the amount of 10³-10¹⁰ Infectious Unit (IU) or 0.01-500 μg per time, preferably 10⁵-10¹⁰ IU or 0.1-100 μg per time, for example 10⁷-10⁹ IU or 1-50 μg per one time. The replicon may preferably be formulated in a vaccine composition suitable for administration in a conventional manner. The present disclosure provides a vaccine composition comprising the alphavirus replicon disclosed herein.

By “subject” is meant a mammal, including, but not limited to, a human or non-human mammal, such as a bovine, equine, canine, ovine, or feline.

As used herein, the terms “treat,” treating,” “treatment,” and the like refer to reducing or ameliorating a disorder and/or symptoms associated therewith. It will be appreciated that, although not precluded, treating a disorder or condition does not require that the disorder, condition or symptoms associated therewith be completely eliminated.

As used herein, the terms “prevent,” “preventing,” “prevention,” “prophylactic treatment” and the like refer to reducing the probability of developing a disorder or condition in a subject, who does not have, but is at risk of or susceptible to developing a disorder or condition.

Unless specifically stated or obvious from context, as used herein, the term “or” is understood to be inclusive.

Unless specifically stated or obvious from context, as used herein, the terms “a”, “an”, and “the” are understood to be singular or plural.

The art will acknowledge that polynucleotide sequences described in the specification and claims will recite “T”s in a representative DNA sequence but where the sequence represents RNA, the “T”s would be substituted for “U”s.

Any vaccine compositions or methods provided herein can be combined with one or more of any of the other vaccine compositions and methods provided herein.

The term “vector” refers to the means by which a nucleic acid sequence can be propagated and/or transferred between organisms, cells, or cellular components. Vectors include plasmids, viruses, bacteriophages, pro-viruses, phagemids, transposons, artificial chromosomes, and the like, that replicate autonomously or can integrate into a chromosome of a host cell. A vector can also be a naked RNA polynucleotide, a naked DNA polynucleotide, a polynucleotide composed of both DNA and RNA within the same strand, a poly-lysine-conjugated DNA or RNA, a peptide-conjugated DNA or RNA, a liposome-conjugated DNA, or the like, that is not autonomously replicating. In many, but not all, common embodiments, the vectors of the present disclosure are plasmids or bacmids.

Typically, the nucleic acid molecule to be expressed is “operably linked” to a promoter and/or enhancer, and is subject to transcription regulatory control by the promoter and/or enhancer.

The method of transfection and the choice of expression vehicle will depend on the host system selected.

Transfection methods are described, e.g., in Ausubel et al. (supra); expression vehicles may be chosen from those provided, e.g., in Cloning Vectors: A Laboratory Manual (P. H. Pouwels et al., 1985, Supp. 1987) The references cited in this paragraph are herein incorporated by reference.

A variety of expression systems exist for the production of the constructs of this disclosure. Expression vectors useful for producing the constructs include, without limitation, chromosomal, episomal, and virus-derived vectors, e.g., vectors derived from bacterial plasmids, from bacteriophage, from transposons, from yeast episomes, from insertion elements, from yeast chromosomal elements, from viruses such as alphavirus (e.g. Chikungunya Virus (CHIKV) and Venezuelan Equine Encephalitis Virus (VEEV)), baculoviruses, papova viruses, such as SV40, vaccinia viruses, adenoviruses, fowl pox viruses, pseudorabies viruses and retroviruses, and vectors derived from combinations thereof.

Constructs and/or vectors used herein comprise alphavirus polynucleotides encoding nonstructural proteins nsp1, nsp2, nsp3 and nsp4 and a gene of interest encoding a polypeptide comprising a coronavirus structural protein fused to a signal sequence and/or a transmembrane domain as discussed above.

The vector may be, for example, a phage, plasmid, viral, or retroviral vector. The constructs and/or vectors that comprise the nucleotides should be operatively linked to an appropriate promoter, such as the CMV promoter, phage lambda PL promoter, the E. coli lac, phoA and tac promoters, the SV40 early and late promoters, and promoters of retroviral LTRs are non-limiting examples. Other suitable promoters will be known to the skilled artisan depending on the host cell and/or the rate of expression desired. The expression constructs will further contain sites for transcription initiation, termination, and, in the transcribed region, a ribosome-binding site for translation. The coding portion of the transcripts expressed by the constructs will preferably include a translation initiating codon at the beginning and a termination codon appropriately positioned at the end of the polypeptide to be translated.

Vectors will preferably include at least one selectable marker. Such markers include dihydrofolate reductase, G418 or neomycin resistance for eukaryotic cell culture and tetracycline, kanamycin or ampicillin resistance genes for culturing in E. coli and other bacteria. Among vectors preferred are virus vectors, such as baculovirus, poxvirus (e.g., vaccinia virus, avipox virus, canarypox virus, fowlpox virus, raccoonpox virus, swinepox virus, etc.), adenovirus (e.g., canine adenovirus), herpesvirus, and retrovirus. Other vectors that can be used with this disclosure comprise vectors for use in bacteria, which comprise pQE70, pQE60 and pQE-9, pBluescript vectors, Phagescript vectors, pNH8A, pNH16a, pNH18A, pNH46A, ptrc99a, pKK223-3, pKK233-3, pDR540, pRIT5. Among preferred eukaryotic vectors are pFastBacl pWINEO, pSV2CAT, pOG44, pXTl and pSG, pSVK3, pBPV, pMSG, and pSVL. Other suitable vectors will be readily apparent to the skilled artisan.

Recombinant constructs can be prepared and used to transfect, can express viral proteins, including those described herein, into eukaryotic cells and/or prokaryotic cells. Thus, in one embodiment, the present disclosure provides host cells which comprise a vector (or vectors) that contain nucleic acids which encode alphavirus structural proteins, including capsid, E3, E2, 6K, and El or portions thereof, and a vector that comprises nucleic acids which encode alphavirus nsp1, nsp2, nsp3 and nsp4, and a gene of interest which encodes a polypeptide comprising a signal sequence, coronavirus RBD, a transmembrane domain and at least one peptide selected from CD4+ T cell epitopes and CD8+ T cell epitopes under conditions which allow the formation of alphavirus replicon particle.

In one embodiment, said vector is a recombinant baculovirus. In another embodiment, said recombinant baculovirus is transfected into an insect cell. In a preferred embodiment, said cell is an insect cell. In another embodiment, said insect cell is a Sf9 cell.

One particular bacterial expression system for polypeptide production is the E. coli pET expression system (Novagen, Inc., Madison, Wis.). According to this expression system, DNA encoding a polypeptide is inserted into a pET vector in an orientation designed to allow expression. Since the gene encoding such a polypeptide is under the control of the T7 regulatory signals, expression of the polypeptide is achieved by inducing the expression of T7 RNA polymerase in the host cell. This is typically achieved by using host strains that express T7 RNA polymerase in response to IPTG induction. Once produced, a recombinant polypeptide is then isolated according to standard methods known in the art, for example, those described herein.

Depending on the vectors and host cells selected, the constructs are produced by growing host cells transfected by the vectors under conditions whereby the recombinant proteins are expressed and the alphavirus replicon is generated, and constructs containing alphavirus replicon being packaged with the particle of alphavirus structural proteins are formed. In one embodiment, provided is a method of producing a construct, that involves co-transfecting a vector comprising a polynucleotide encoding alphavirus non-structural protein nsp1, nsp2, nsp3 and nsp4, and at least one gene of interest encoding the polypeptide comprising a coronavirus structural protein fused to a signal sequence and/or a transmembrane domain, and at least one vector each encoding at least one alphavirus structural protein into suitable host cells and expressing said alphavirus structural protein under conditions that allow construct formation. In another embodiment, the eukaryotic cell is selected from the group consisting of, yeast, insect, amphibian, avian or mammalian cells. The selection of the appropriate growth conditions is within the skill or a person with skill of one of ordinary skill in the art.

Methods to grow cells that produce alphavirus replicon particles of the disclosure include, but are not limited to, batch, batch-fed, continuous and perfusion cell culture techniques. In one embodiment, cells co-transfected with a vector encoding an alphavirus replicon and a vector comprising a polypeptide encoding capsid, and a vector comprising a polynucleotide encoding envelope proteins, such as those derived from a CHIKV or VEEV are grown in a bioreactor or fermentation chamber where cells propagate and express protein (e.g., recombinant proteins) for purification and isolation. Typically, cell culture is performed under sterile, controlled temperature and atmospheric conditions. A bioreactor is a chamber used to culture cells in which environmental conditions such as temperature, atmosphere, agitation and/or pH can be monitored. In one embodiment, the bioreactor is a stainless steel chamber. In another embodiment, said bioreactor is a pre-sterilized plastic bag (e.g., Cellbag™, Wave Biotech, Bridgewater, N.J., the contents of the cited document is herein incorporated by reference). In other embodiment, said pre-sterilized plastic bags are about 10 L to 1000 L bags.

In another embodiment, an RNA molecule such as an alphavirus replicon may be generated by conventional procedures known to the art from a template DNA sequence. In vitro transcription (IVT) methods permit template-directed synthesis of RNA molecules. IVT methods permit synthesis of large quantities of RNA transcript. Generally, IVT utilizes a DNA template comprising a promoter sequence upstream of a sequence of interest. The promoter sequence is most commonly of bacteriophage origin such as the T7, T3 or SP6 promoter sequence but many other promotor sequences can be tolerated including those designed de novo. Transcription of the DNA template is typically best achieved by using the RNA polymerase corresponding to the specific bacteriophage promoter sequence. Exemplary RNA polymerases include, but are not limited to T7 RNA polymerase, T3 RNA polymerase, or SP6 RNA polymerase, among others. IVT is generally initiated at a dsDNA but can proceed on a single strand. Kits for in vitro transcription such as T7 transcription kit (RiboMax™ Express Large Scale RNA production System, Promega (WI USA)).

As used herein, the term “pharmaceutically acceptable carrier” means one or more compatible solid or liquid fillers, diluents or encapsulating substances which are suitable for administration to a human or other vertebrate animal, including any and all aqueous solvents (e.g., water, alcoholic/aqueous solutions, saline solutions, parenteral vehicles, such as sodium chloride, and Ringer's dextrose), non-aqueous solvents (e.g., propylene glycol, polyethylene glycol, vegetable oil, and injectable organic esters, such as ethyloleate), dispersion media, coatings, surfactants, antioxidants, preservatives (e.g., antibacterial or antifungal agents, anti-oxidants, chelating agents, and inert gases), isotonic agents, absorption delaying agents, salts, drugs, drug stabilizers, gels, binders, excipients, disintegration agents, lubricants, sweetening agents, flavoring; agents, dyes, fluid and nutrient replenishers, such like materials and combinations thereof, as would be known to one of ordinary skill in the art. The pH and exact concentration of the various components in a vaccine composition are adjusted according to well-known parameters.

Encapsulating substances refers to a delivery vehicle in which the polynucleotide or vector is packaged, such as a replicon particle (e.g. the alphavirus replicon particle described in US patent publication. No. 2019/0185822, the contents of the document is incorporated by reference) and a lipid delivery system (e.g. liposome).

In some embodiments, the vaccine compositions or formulations of the present disclosure comprise a lipid delivery system, e.g., a liposome, a lioplexes, a lipid nanoparticle, or any combination thereof. The polynucleotides such as an alpha virus replicon described herein can be formulated using one or more liposomes, lipoplexes, or lipid nanoparticles. Liposomes, lipoplexes, or lipid nanoparticles can be used to improve the efficacy of the polynucleotides directed protein production as these formulations can increase cell transfection by the polynucleotide; and/or increase the translation of encoded protein. The liposomes, lipoplexes, or lipid nanoparticles can also be used to increase the stability of the polynucleotides.

Liposomes are artificially-prepared vesicles that may primarily be composed of a lipid bilayer and may be used as a delivery vehicle for the administration of pharmaceutical formulations. Liposomes can be of different sizes. A multilamellar vesicle (MLV) may be hundreds of nanometers in diameter, and may contain a series of concentric: bilayers separated by narrow aqueous compartments. A small unicellular vesicle (SUV) may be smaller than 50 nm in diameter, and a large unilamellar vesicle (LUV) may be between 50 and 500 nm in diameter. Liposome design may include, but is not limited to, opsonins or ligands to improve the attachment of liposomes to unhealthy tissue or to activate events such as, but not limited to, endocytosis. Liposomes may contain a low or a high pH value in order to improve the delivery of the pharmaceutical formulations.

The formation of liposomes may depend on the pharmaceutical formulation entrapped and the liposomal ingredients, the nature of the medium in which the lipid vesicles are dispersed, the effective concentration of the entrapped substance and its potential toxicity, any additional processes involved during the application and/or delivery of the vesicles, the optimal size, polydispersity and the shelf-life of the vesicles for the intended application, and the batch-to-batch reproducibility and scale up production of safe and efficient liposomal products, etc.

In some embodiments, the polynucleotides such as alpha virus replicon described herein may be encapsulated by the liposome and/or it may be contained in an aqueous core that may then be encapsulated by the liposome.

In some embodiments, the polynucleotides such as alpha virus replicon described herein can be formulated in a cationic oil-in-water emulsion where the emulsion particle comprises an oil core and a cationic lipid that can interact with the polynucleotide anchoring the molecule to the emulsion particle. In some embodiments, the polynucleotides described herein can be formulated in a water-in-oil emulsion comprising a continuous hydrophobic phase in which the hydrophilic phase is dispersed.

In some embodiments, the polynucleotides such as alpha virus replicon described herein can be formulated in a lipid-polycation complex. As a non-limiting example, the polycation can include a cationic peptide or a polypeptide such as, but not limited to, polylysine, polyornithine and/or polyarginine and the cationic peptides.

In some embodiments, the polynucleotides such as alpha virus replicon described herein can be formulated in a lipid nanoparticle (LNP).

Lipid nanoparticle formulations typically comprise one or more lipids. In some embodiments, the lipid is a cationic or an ionizable lipid. In some embodiments, lipid nanoparticle formulations further comprise other components, including a phospholipid, a structural lipid, a quaternary amine compound, and a molecule capable of reducing particle aggregation, for example a PEG or PEG-modified lipid. In some embodiments, the amount of the cationic and ionizable lipids in the lipid composition ranges from about 0.01 mol % to about 99 mol %.

LNPs contain a pH-sensitive ionizable cationic lipid that attract anionic nucleic acids to form the core of self-assembling nanoparticle to ensure high encapsulation. At physiological pH, IMPS are neutral, eliminating a mechanism of toxicity seen with permanently cationic molecules.

These same pH-sensitive lipids are responsible for responding to the acidic environment of the endosome and triggering the disruption of the endosome and release of the nucleic acid into the cell.

This replicon based vaccine technology is a unique platform technology for the vaccination as a RNA can self-amplify to produce the vaccine antigen and deliver into the cellular organ. Moreover, this replicon based vaccine technology overcomes the challenges commonly associated with DNA based vaccines, such as risk of genome integration or the high doses and devices needed for administration, e.g. electroporation, and expects the higher immunogenicity with minimum dose based on the self-replication system over the mRNA technology. Also, we designed the antigens based on receptor binding domain (RBD) with a signal sequence, a transmembrane domain and at least one peptide selected from CD4+ T cell epitopes and CD8+ T cell epitopes. Preferable embodiment includes a universal epitope for CD4+ T cells such as PADRE to enhance the immunogenicity.

In one embodiment, the vaccine composition may be monovalent, containing only one type of alphavirus replicon. In another embodiment, the vaccine composition is bivalent, comprising two types of alpha virus replicons each encodes an antigenic protein derived from a different strain or variant.

According to the present disclosure, novel antigenically-active proteins/polypeptides are also useful for producing antibodies for diagnosis and protecting against coronaviruses while minimizing the possibility of ADE. The proteins/polypeptides disclosed herein include minimum sequences encoding the coronavirus RBD fused to a signal sequence, a transmembrane domain (TMD) and at least one peptide selected from CD4+ T cell epitopes and CD8+ T cell epitopes, intended to maximize immunogenicity and minimize ADE.

This efficient vaccine design may also be applied to the development of vaccines against a cancer or an inflammatory disease.

The invention will be described in detail with reference to the following examples, which, however, are not intended to limit the scope of the present application.

### Example 1

Gene encoding shown below construct 1 was synthesized by Integrated DNA Technologies, Inc.

(https://www.idtdna.com/pages).

Construct 1

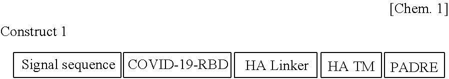

Whole amino acid of construct 1 us as follows:

(SEQ ID NO: 2)
MFVELVLLPLVSSVREPNITNLCPFGEVFNATRFASVYAWNRKRISNCV
ADYSVLYNSASFSTFKCYGVSPTKLNDLCFTNVYADSFVIRGDEVRQIA
PGQTGTIADYNYKLPDDFTGCVIAWNSNNLDSKVGGNYNYLYRLFRKSN
LKPFERDISTEIYQAGSTPCNGVKGFNCYFPLQSYGFQPTYGVGYQPYR
VVVLSFELLHAPATVCGPKKSTGVKLESMGIYQILAIYSTVASSLVLLV
SLGAISFWMCSNGSLQCRICIAKFVAAWTLKAAA

Signal sequence COVID19 (1-13):

(SEQ ID NO: 3
MFVFLVLLPLVSS

COVID-19—RBD (Brazilian (Gamma) variant):

(SEQ ID NO: 4)
VRFPNITNLCPFGEVFNATRFASVYAWNRKRISNCVADYSVLYNSASFS
TFKCYGVSPTKLNDLCFTNVYADSFVIRGDEVRQIAPGQTGTIADYNYK
LPDDFTGCVIAWNSNNLDSKVGGNYNYLYRLFRKSNLKPFERDISTEIY
QAGSTPCNGVKGFNCYFPLQSYGFQPTYGVGYQPYRVVVLSFELLHAPA
TVCGPKKST

HA Linker:

(SEQ ID NO: 5
GVKLESMGIY

HA TM:

(SEQ ID NO: 6)
QILAIYSTVASSLVLLVSLGAISFWMCSNGSLQCRICI

PADRE:

(SEQ ID NO: 1
AKFVAAWTLKAAA

### Example 2

Gene encoding shown below construct 2 and 3 were synthesized by Integrated DNA Technologies, Inc.

(https://www.idtdna.com/pages).

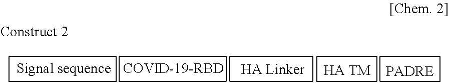

Whole amino acid:

(SEQ ID NO: 7)
MEVELVLLPLVSSVREPNITNLCPFDEVFNATRFASVYAWNRKRISNCV
ADYSVLYNLAPFFTFKCYGVSPTKLNDLCFTNVYADSFVIRGDEVRQIA
PGQTGNIADYNYKLPDDFTGCVIAWNSNKLDSKVSGNYNYLYRLFRKSN
LKPFERDISTEIYQAGNKPCNGVAGFNCYFPLKSYSFRPTYGVGHQPYR
VVVLSFELLHAPATVCGPKKSTGVKLESMGIYQILAIYSTVASSLVLLV
SLGAISFWMCSNGSLQCRICIAKFVAAWTLKAAA*

Signal sequence:

(SEQ ID NO: 3)
MFVFLVLLPLVSS

COVID-19—RBD (Omicron variant):

(SEQ ID NO: 8)
VRFPNITNLCPFDEVFNATRFASVYAWNRKRISNCVADYSVLYNLAPFF
TFKCYGVSPTKLNDLCFTNVYADSFVIRGDEVRQIAPGQTGNIADYNYK
LPDDFTGCVIAWNSNKLDSKVSGNYNYLYRLFRKSNLKPFERDISTEIY
QAGNKPCNGVAGFNCYFPLKSYSFRPTYGVGHQPYRVVVLSFELLHAPA
TVCGPKKST

HA Linker:

(SEQ ID NO: 5)
GVKLESMGIY

HA TM:

(SEQ ID NO: 6)
QILAIYSTVASSLVLLVSLGAISFWMCSNGSLQCRICI

PADRE:

(SEQ ID NO: 1)
AKFVAAWTLKAAA

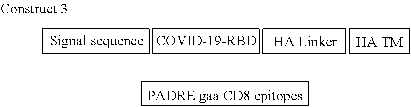

Whole amino acid:

(SEQ ID NO: 9)
MEVELVLLPLVSSVREPNITNLCPFDEVFNATRFASVYAWNRKRISNCV
ADYSVLYNLAPFFTFKCYGVSPTKLNDLCFTNVYADSFVIRGDEVRQIA
PGQTGNIADYNYKLPDDFTGCVIAWNSNKLDSKVSGNYNYLYRLFRKSN
LKPFERDISTEIYQAGNKPCNGVAGFNCYFPLKSYSFRPTYGVGHQPYR
VVVLSFELLHAPATVCGPKKSTGVKLESMGIYQILAIYSTVASSLVLLV
SLGAISFWMCSNGSLQCRICIAKFVAAWTLKAAAgaaVYFLQSINFNAA
SPRWYFYYLNARLNEVAKNLKFIAGLIAIVNAAANFKDQVILLGAAAVL
QSGFRKGAAAYYQLYSTQLNLITGRLQSLKLQLPQGTTLNAAKTFPPTE
PKKAAGDAALALLLLNAAAMEVTPSGTWLGAGVAMPNLYKKAATLACFV
LAAVNAAGLMWLSYFIGLWLLWPVTLNKQFDTYNLW*

Linker:

gaa

CD8 epitopes:

(SEQ ID NO: 10)
VYFLQSINFNAASPRWYFYYLNARLNEVAKNLKFIAGLIAIVNAAANFK
DQVILLGAAAVLQSGFRKGAAAYYQLYSTQLNLITGRLQSLKLQLPQGT
TLNAAKTFPPTEPKKAAGDAALALLLLNAAAMEVTPSGTWLGAGVAMPN
LYKKAATLACFVLAAVNAAGLMWLSYFIGLWLLWPVTLNKQFDTYNLW

### Example 3

### Preparation of Replicon Vector

Schematic construct of the alphavirus replicon is shown in FIG. 1.

Gene of Interest DNA prepared in Example 1 was cloned into the VEEV replicon vector under the control of the subgenomic (SG) promoter. The VEEV replicon plasmid encoding each fragment was created by insertion at AscI and SbfI restriction sites to obtain the full-length VEEV TC-83 replicon construct.

Nucleotide sequences of SG promoter, 5′UTR, 3′UTR and Poly A tail are as follows. RNA sequences were obtained by using those DNA sequences as template.

SG promoter:

(SEQ ID NO: 11)
cctgaatggactacgacatagtctagtccgccaag

5′UTR:

(SEQ ID NO: 12)
ataggcggcgcatgagagaagcccagaccaattacctacccaaa

3′UTR:

(SEQ ID NO: 13)
gcgatcgcatacagcagcaattggcaagctgcttacatagaactcgcgg
cgattggcatgccgccttaaaatttttattttatttttcttttcttttc
cgaatcggattttgtttttaatatttc

Poly A tail:

(SEQ ID NO: 14)
aaaaaaaaaaaaaaaaaaaaaaaaaaaaaaaaaaaaaaaaaaaaaaaaa
aaaaaa

VEEV TC-83 Replicon nsP1-4 amino acid sequence is as follows.

(SEQ ID NO: 15)
MEKVHVDIEEDSPFLRALQRSFPQFEVEAKQVTDNDHANARAFSHLASK
LIETEVDPSDTILDIGSAPARRMYSKHKYHCICPMRCAEDPDRLYKYAT
KLKKNCKEITDKELDKKMKELAAVMSDPDLETETMCLHDDESCRYEGQV
AVYQDVYAVDGPTSLYHQANKGVRVAYWIGFDTTPFMFKNLAGAYPSYS
TNWADETVLTARNIGLCSSDVMERSRRGMSILRKKYLKPSNNVLFSVGS
TIYHEKRDLLRSWHLPSVFHLRGKQNYTCRCETIVSCDGYVVKRIAISP
GLYGKPSGYAATMHREGFLCCKVTDTLNGERVSFPVCTYVPATLCDOMT
GILATDVSADDAQKLLVGLNQRIVVNGRTQRNTNTMKNYLLPVVAQAFA
RWAKEYKEDQEDERPLGLRDRQLVMGCCWAFRRHKITSIYKRPDTQTII
KVNSDFHSFVLPRIGSNTLEIGLRTRIRKMLEEHKEPSPLITAEDIQEA
KCAADEAKEVREAEELRAALPPLAADFEEPTLEADVDLMLQEAGAGSVE
TPRGLIKVTSYAGEDKIGSYAVLSPQAVLKSEKLSCIHPLAEQVIVITH
SGRKGRYAVEPYHGKVVVPEGHAIPVQDFQALSESATIVYNEREFVNRY
LHHIATHGGALNTDEEYYKTVKPSEHDGEYLYDIDRKQCVKKELVTGLG
LTGELVDPPFHEFAYESLRTRPAAPYQVPTIGVYGVPGSGKSGIIKSAV
TKKDLVVSAKKENCAEIIRDVKKMKGLDVNARTVDSVLLNGCKHPVETL
YIDEAFACHAGTLRALIAIIRPKKAVLCGDPKQCGFFNMMCLKVHFNHE
ICTQVFHKSISRRCTKSVTSVVSTLFYDKRMRTTNPKETKIVIDTTGST
KPKQDDLILTCFRGWVKQLQIDYKGNEIMTAAASQGLTRKGVYAVRYKV
NENPLYAPTSEHVNVLLTRTEDRIVWKTLAGDPWIKILTAKYPGNFTAT
IEEWQAEHDAIMRHILERPDPTDVFQNKANVCWAKALVPVLKTAGIDMT
TEQWNTVDYFETDKAHSAEIVLNQLCVRFEGLDLDSGLFSAPTVPLSIR
NNHWDNSPSPNMYGLNKEVVRQLSRRYPQLPRAVATGRVYDMNTGTLRN
YDPRINLVPVNRRLPHALVLHHNEHPQSDFSSFVSKLKGRTVLVVGEKL
SVPGKKVDWLSDQPEATFRARLDLGIPGDVPKYDIVFINVRTPYKYHHY
QQCEDHAIKLSMLTKKACLHLNPGGTCVSIGYGYADRASESIIGAIARQ
FKFSRVCKPKSSHEETEVLFVFIGYDRKARTHNPYKLSSTLTNIYTGSR
LHEAGCAPSYHVVRGDIATATEGVIINAANSKGQPGGGVCGALYKKFPE
SFDLQPIEVGKARLVKGAAKHIIHAVGPNFNKVSEVEGDKQLAEAYESI
AKIVNDNNYKSVAIPLLSTGIFSGNKDRLTQSLNHLLTALDTTDADVAI
YCRDKKWEMTLKEAVARREAVEEICISDDSSVTEPDAELVRVHPKSSLA
GRKGYSTSDGKTFSYLEGTKFHQAAKDIAEINAMWPVATEANEQVCMYI
LGESMSSIRSKCPVEESEASTPPSTLPCLCIHAMTPERVQRLKASRPEQ
ITVCSSFPLPKYRITGVQKIQCSQPILFSPKVPAYIHPRKYLVETPPVE
ETPESPAENQSTEGTPEQPALVNVDATRTRMPEPIIIEEEEEDSISLLS
DGPTHQVLQVEADIHGSPSVSSSSWSIPHASDFDVDSLSILDTLDGASV
TSGAVSAETNSYFARSMEFRARPVPAPRTVFRNPPHPAPRTRTPPLAHS
RASSRTSLVSTPPGVNRVITREELEALTPSRAPSRSASRTSLVSNPPGV
NRVITREEFEAFVAQQQXRFDAGAYIFSSDTGQGHLQQKSVRQTVLSEV
VLERTELEISYAPRLDQEKEELLRKKLQLNPTPANRSRYQSRRVENMKA
ITARRILQGLGHYLKAEGKVECYRTLHPVPLYSSSVNRAFSSPKVAVEA
CNAMLKENFPTVASYCIIPEYDAYLDMVDGASCCLDTASFCPAKLRSFP
KKHSYLEPTIRSAVPSAIQNTLQNVLAAATKRNCNVTQMRELPVLDSAA
FNVECFKKYACNNEYWETFKENPIRLTEENVVNYITKLKGPKAAALFAK
THNLNMLQDIPMDRFVMDLKRDVKVTPGTKHTEERPKVQVIQAADPLAT
ADLCGIHRELVRRLNAVLLPNIHTLFDMSAEDFDAIIAEHFQPGDCVLE
TDIASFDKSEDDAMALTALMILEDLGVDAELLTLIEAAFGEISSIHLPT
KTKFKFGAMMKSGMFLTLFVNTVINIVIASRVLRERLTGSPCAAFIGDD
NIVKGVKSDKLMADRCATWLNMEVKIIDAVVGEKAPYFCGGFILCDSVT
GTACRVADPLKRLFKLGKPLAVDDEHDDDRRRALHEESTRWNRVGILPE
LCKAVESRYETVGTSIIVMAMTTLASSVKSFSYLRGAPITLYG.

Amino acid sequence corresponding to nsp3 is underlined.

### Example 4

### Preparation of Self-Amplifying RNA (saRNA) Encapsulated in Lipid Nanoparticles (LNP)

The vectors comprising the DNA sequence encoding construct 1 to 3 prepared in Examples 1 and 2 were used. The DNA was linearized and used as the template. T7 in vitro transcription was conducted based on protocols provided by the T7 transcription kit (RiboMax™ Express Large Scale RNA production System, Promega, (WI USA)). The linear DNA template was mixed with T7 enzyme and rNTPs to synthesize RNA. For the synthesis of RNA containing a modified nucleotide, a modified NTP such as 5-methyl-cytidine and N1-methyl-pseudouridine triphosphate was added to the in vitro transcription reaction mixture. The purified RNA product was capped using vaccinia capping enzyme to give self-amplifying RNA.

In the same manner as above, a saRNA comprising a gene of interest encoding a control construct “RBD(Wuhan)-TM” consisting of COVID19 signal sequence, COVID-19-RBD of Wuhan strain, HA linker and HA TM, and also a saRNA comprising a gene of interest encoding a construct “RBD(Brazil)-TM” consisting of COVID19 signal sequence, COVID-19-RBD of Brazilian(gamma) variant, HA linker and HA TM.

The amino acid sequence of “RBD(Wuhan)-TM” is as follows:

(SEQ ID NO: 16)
MEVELVLLPLVSSVREPNITNLCPFGEVFNATRFASVYAWNRKRISNCV
ADYSVLYNSASFSTFKCYGVSPTKLNDLCFTNVYADSFVIRGDEVRQIA
PGQTGKIADYNYKLPDDFTGCVIAWNSNNLDSKVGGNYNYLYRLFRKSN
LKPFERDISTEIYQAGSTPCNGVEGFNCYFPLQSYGFQPTNGVGYQPYR
VVVLSFELLHAPATVCGPKKSTGVKLESMGIYQILAIYSTVASSLVLLV
SLGAISFWMCSNGSLQCRICI

In the above, the amino acid sequence of COVID-19-RBD of Wuhan strain is as follows:

(SEQ ID NO: 17)
VRFPNITNLCPFGEVFNATRFASVYAWNRKRISNCVADYSVLYNSASFS
TFKCYGVSPTKLNDLCFTNVYADSFVIRGDEVRQIAPGQTGKIADYNYK
LPDDFTGCVIAWNSNNLDSKVGGNYNYLYRLFRKSNLKPFERDISTEIY
QAGSTPCNGVEGFNCYFPLQSYGFQPTNGVGYQPYRVVVLSFELLHAPA
TVCGPKKST

The obtained saRNAs were encapsulated in lipid nanoparticles to give saRNA particles. Obtained saRNA particles were used in the following examples.

### Example 5

### Immunization

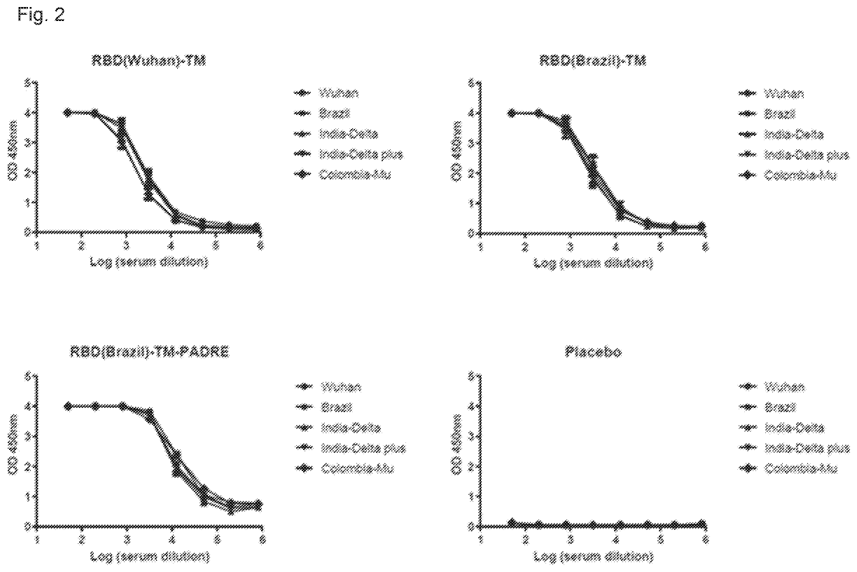

Methods: Golden syrian hamsters (n=12 per group) were immunized intramuscularly twice with 10 ug of saRNA LNP expressing SARS-CoV-2 Spike RBD (Wuhan strain) with HA transmembrane (RBD(Wuhan)-TM), RBD Brazil (Gamma) with HA transmembrane (RBD(Brazil)-TM) and construct 1 of example 1, i.e RBD (Brazil (Gamma)) with HA transmembrane plus PADRE (RBD(Brazil)-TM-PADRE) or PBS (Placebo) at 0 and 4 weeks. Antibody titers of sera from the immunized hamsters on 4 weeks after 2nd immunization (Day 55) were evaluated by ELISA against SARS-CoV-2RBD proteins of Wuhan strain, Brazilian variant, India-Delta variant, India-Delta plus variant and Colombia-Mu variant. Results are shown in FIG. 2.

Results: The antibodies in the sera immunized with RBD(Brazil (Gamma))-TM-PADRE induced more than 3 times higher antibodies (IC50 titer) against each RBD variant compared to the antibodies in the sera immunized with RBD(Brazil (Gamma))-TM.

### Example 6

### ACE2 Inhibition Assay

Method: Using commercially available kit (Genscript L00847) inhibitory effects of serum obtained from hamsters immunized with the vaccines prepared in Example 4 on the binding between RBD variants and ACE2 were examined. Serum were incubated with RBD variants conjugated to horse-radish-peroxidase at 37° C. for 30 min. The mixture were added to an ACE2-coated 96 well plate and incubated at 37° C. for 15 min. After washing the plate, TMB substrate was added and incubated at room temperature for 15 min. Absorbance at 450 nm was measured in microplate reader. From the absorbance the inhibition was calculated according to the formula below.

inhibition(%)=(1−absorbance of sample/absorbance of average of negative control)×100

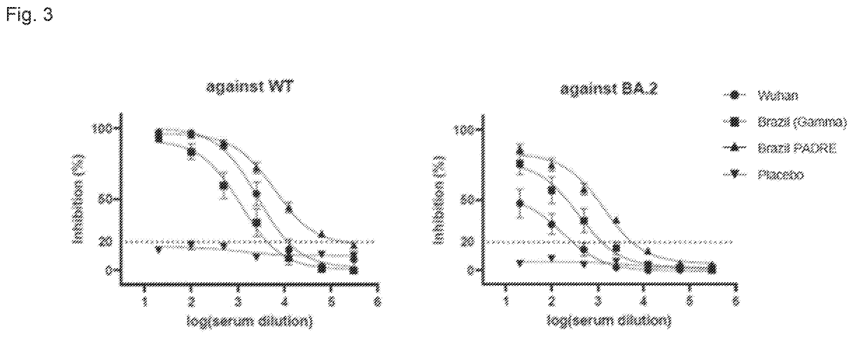

Results are shown in FIG. 3.

Results: All vaccines have inhibitory effect against SARS-CoV-2 Omicron BA. 2 variant as well as Wuhan strain, and Brazil Gamma PADRE vaccine is a highest inhibitory effect among them.

### Example 7

### Effect of Modified Nucleoside in Innate Immunity

Methods: THP-1 Dual cell (InvivoGen) in which a secreted luciferase (Luca gene) is featured under the control of an minimal promoter of interferon-stimulated gene (ISG) 54 in conjunction with IFN-Stimulated Response Elements (ISRE) were used. The IFN regulatory factor (IRF) inducing effects of Construct 1 of Example 1 (Sample 1) and Construct 1 of Example 1 provided that 5-methyl-cytidine was used instead of cytidine (Sample 2) were examined. Passaged THP-1 Dual cells were harvested and prepared 5.6×105 cells/mL of cell suspension with culture medium (RPMI-1640+10% FBS). 180 μL of this cell suspension was seeded into each well of a 96-well culture plate. Then, 20 μL of phosphate-buffered saline (control) or a mixture of Lipofectamine (Lipofectamine™ RNAiMAX Transfection Reagent) and Sample 1 or Sample 2 were added per well. The cells were incubated at 37° C. under 5% CO2 for 24 hours.

20 μL of the culture supernatant was aliquoted into another 96-well white plate, 50 μL of the effervescent reagent (QUANTI-Luc, InvivoGen) solution was added to each well, and the luminescence intensity was measured immediately after addition.

From the luminescence intensity, the fold increase of each sample was calculated according to the formula below and used as an index of IRF induction.

Fold increase=(luminescence intensity of sample)/(luminescence intensity of control)

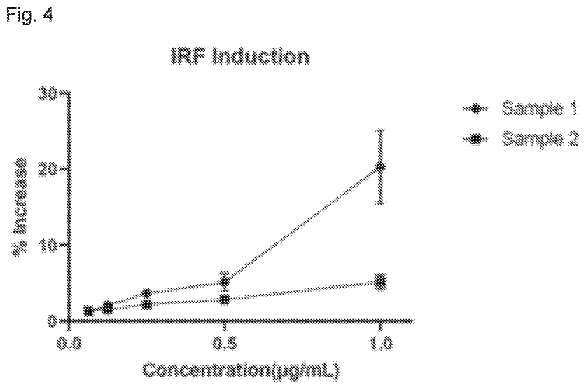

The result is shown in FIG. 4.

Compared to Sample 1 (using cytidine), the IRF induction of Sample 2 (using 5-methyl-cytidine, modified nucleoside) was lower, showing a clear reduction of the innate immunity stimulating effect.

### Example 8

### Animal Study

Golden Syrian Hamsters (N=6 per group) were injected intramuscularly twice with 10 ug of saRNA prepared in Example 4. The saRNAs used in this example were RBD(Wuhan)-TM, RBD(Brazil)-TM, and RBD(Brazil)-TM-PADRE at 0 and 4 weeks. Hamsters were challenged with live SARS CoV-2 Wuhan strain (WT) or Brazilian (Gamma) variant at Day 56.

Body Weight Measurements

Methods:

Body weights were recorded daily post challenge, and the mean percentages of body weight change, from study day 56 to termination on study day 64.

Results:

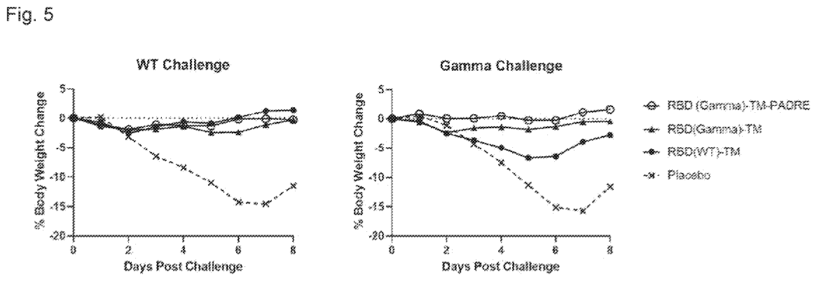

Results are shown in FIG. 5. The group immunized with RBD(Gamma)-TM-PADRE prevented weight loss of the hamsters to the similar level to RBD(Gamma)-TM or RBD(WT)-TM immunized groups in WT challenged hamsters (left panel). On the other hand, no reduction of weight was observed in RBD(Gamma)-TM-PADRE in Gamma challenged animals, while other two groups exhibited mild reduction (right panel).

Quantitative RT-PCR Assay for SARS-CoV-2

Methods:

Oral swabs were collected from anesthetized animals on select time points post-challenge and tested for amounts of SARS-CoV2 RNA copies per mL. The lower detection limit for this assay is 50 RNA copies/mL.

Results:

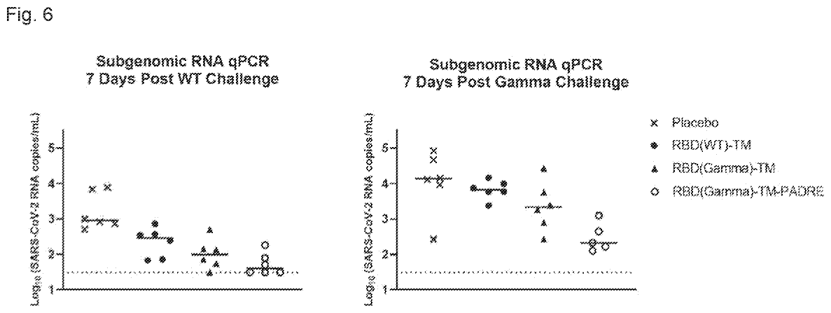

Results are shown in FIG. 6. A greater reduction in oral swab viral RNA loads were observed in RBD(Gamma)-TM-PADRE immunized group compared to RBD(Gamma)-TM or RBD(WT)-TM immunized groups after WT (left panel) or Gamma (right panel) challenge.

Histology

Methods:

At necropsy, left lung was collected and placed in 10% neutral buffered formalin for histopathologic analysis. Tissue sections were trimmed and processed to hematoxylin and eosin (H&E) stained slides.

Results:

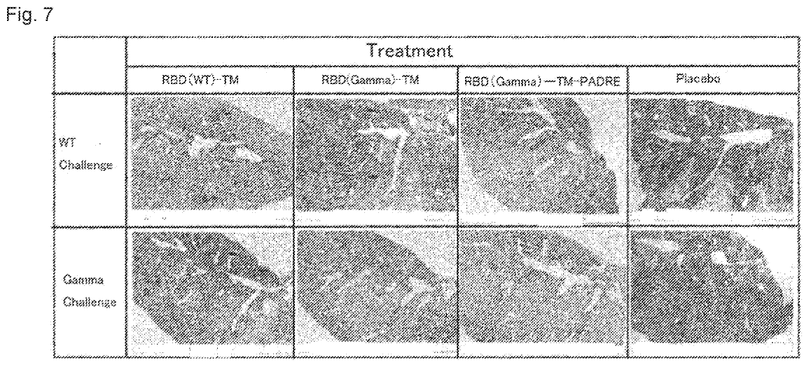

The results are shown in FIG. 7. The group Immunized with RBD(Gamma)-TM-PADRE prevented development of pathological findings in the lung tissues after WT (The third column from left, top) or Gamma (The third column from left, bottom) challenge

What is claimed is:

1. An isolated polynucleotide, which encodes alphavirus non-structural proteins nsp1, nsp2, nsp3 and nsp4 and a polypeptide comprising an antigenic protein fused to a signal sequence, a transmembrane domain and at least one peptide selected from CD4+ T cell epitopes and CD8+ T cell epitopes.

2. The polynucleotide of claim 1, wherein the antigenic protein is fused to a CD4+ T cell epitope, and wherein the CD4+ T cell epitope is a Pan-DR epitope (PADRE).

3. The polynucleotide of claim 1, wherein the antigenic protein is a protein derived from a virus, a cancer or cytokine.

4. The polynucleotide of claim 1, wherein the antigenic protein is a protein derived from a virus or bacterium selected from the group consisting of Severe acute respiratory syndrome-related coronavirus (SARS), severe acute respiratory syndrome coronavirus 2 (SARS-CoV-2), Ebola virus, HIV, Hepatitis B virus (HBV), influenza virus, Hepatitis C virus (HCV), Human papillomavirus (HPV), Cytomegalovirus (CMV), Chikungunya virus, Respiratory syncytial virus (RSV), Dengue virus, a orthymyxoviridae family virus, and Mycobacterium tuberculosis.

5. The polynucleotide of claim 4, wherein the antigenic protein is further fused to a CD8+ T cell epitope, and wherein the CD8+ T cell epitope is a peptide derived from the virus from which the antigenic protein is derived.

6. The polynucleotide of claim 4, wherein the antigenic protein is derived from a coronavirus.

7. The polynucleotide of claim 6, wherein the antigenic protein is a receptor binding domain (RBD) of the coronavirus S1 subunit.

8. The polynucleotide of claim 1, wherein the transmembrane domain is derived from Influenza Hemagglutinin (HA), CD80, or a modified transmembrane domain derived from the antigenic protein.

9. The polynucleotide of claim 8, wherein the transmembrane domain is derived from Influenza Hemagglutinin (HA).

10. The polynucleotide of claim 8, wherein the modified transmembrane domain comprises juxtamembrane domain and transmembrane domain of COVID-19 Spike (S) protein.

11. The polynucleotide of claim 6, wherein the coronavirus is COVID-19.

12. A vector comprising the polynucleotide of claim 1.

13. The vector of claim 12, which comprises a promoter, 5′ UTR, a polynucleotide encoding alphavirus non-structural proteins nsp1, nsp2, nsp3 and nsp4, a SG promoter, a gene of interest encoding a polypeptide comprising an antigenic protein which is fused to a signal sequence, a transmembrane domain and at least one peptide selected from CD4+ T cell epitopes and CD8+ T cell epitopes, 3′UTR and poly A tail.

14. A vaccine composition comprising the polynucleotide of claim 1 or a vector comprising the polynucleotide of claim 1, and a pharmaceutically acceptable delivery vehicle.

15. A method of treating, preventing and/or immunizing against an antigen in a subject, comprising administering an effective amount of the vaccine of claim 14 to the subject in need thereof.

16. An isolated polynucleotide, which encodes alphavirus non-structural proteins nsp1, nsp2, nsp3 and nsp4 and a polypeptide comprising an antigenic protein fused to a signal sequence, a transmembrane domain and at least one peptide selected from CD4+ T cell epitopes and CD8+ T cell epitopes, wherein the polynucleotide comprises a modified nucleoside.

17. The polynucleotide of claim 16, wherein the modified nucleoside is modified cytidine and/or modified uridine.

18. The polynucleotide of claim 17, wherein the modified cytidine is 5-methyl-cytidine and the modified uridine is N1-methyl-pseudouridine.

19. The polynucleotide of claim 17, wherein substantially 100% of cytidine in the polynucleotide are modified cytidine.

20. The polynucleotide of claim 17, wherein the less than 100% of uridine in the polynucleotide are modified uridine.
